(19) United States
(12) Patent Application Publication
(10) Pub. No.: US 2008/0260678 A1
(43) Pub. Date: Oct. 23, 2008

(54) MOLECULAR BAND-AID

(75) Inventors: BRIAN TSENG, BOULDER, CO (US); RICK RABON, BOULDER, CO (US); LALITH POLEPEDDI, Centennial, CO (US); MICHAEL M. POLMEAR, Greenwood Village, CO (US); WARREN MCCLURE, Denver, CO (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: UNIVERSITY OF COLORADO, BOULDER, CO (US)

(21) Appl. No.: 11/869,908

(22) Filed: Oct. 10, 2007

Related U.S. Application Data
(60) Provisional application No. 60/850,654, filed on Oct. 10, 2006.

(51) Int. Cl. A61K 31/74; A61P 21/00

(52) U.S. Cl. 424/78.31

(57) ABSTRACT

A molecular sealant comprising a therapeutically effective amount of a poloxamer capable of sealing a cell or tissue from leaks. A method of treating tissue leakages by applying the above sealant to the tissue or cell in need of treatment. A composition for treating cellular leakages comprising a poloxamer capable of sealing a cell or tissue from leaks in a pharmaceutically acceptable carrier.

### CROSS-REFERENCE TO RELATED APPLICATIONS

This application claims the benefit of priority to U.S. Provisional Patent Application Ser. No. 60/850,654, filed Oct. 10, 2006, incorporated herein in its entirety.

### BACKGROUND OF THE INVENTION

1. Technical Field

The present invention relates to a product and method for treating muscular dystrophy. More specifically, the present invention relates to a poloxamer for use in treating membrane leakages.

2. Description of the Related Art

Striated skeletal muscles are highly ordered structures arranged for active displacement and force generation. The information transmission in skeletal muscle, as in other excitable cells, depends on electrical currents flowing across the cell membrane. In the resting state, high K⁺ permeability is responsible for the setting of membrane potential, and the activities of K⁺ channels are regulated by a wide range of cellular mediators, such as ions, nucleotides, and lipids. Alterations in the currents can effect the permeability of the cell membrane, thereby allowing cellular components to leak from the interior of the cell. If such leakage occurs repeatedly, it results in a disease state.

One example of such as disease state is Duchenne Muscular Dystrophy (DMD). DMD is a rapidly progressive form of muscular dystrophy. It is characterized by progressive muscle weakness of the legs and pelvis which is associated with a loss of muscle mass (wasting). Muscle weakness also occurs in the arms, neck, and other areas, but not as severely as in the lower half of the body. Calf muscles initially enlarge (an attempt by the body to compensate for loss of muscle strength), but the enlarged muscle tissue is eventually replaced by fat and connective tissue (pseudohypertrophy). Muscle contractions occur in the legs and heels, causing inability to use the muscles because of shortening of muscle fibers and fibrosis of connective tissue. Bones develop abnormally, causing skeletal deformities of the chest and other areas. Cardiomyopathy occurs in almost all cases. Mental retardation may accompany the disorder but it is not inevitable and does not worsen as the disorder progresses. The cause of this impairment is unknown.

DMD occurs in approximately 1 out of 3,500 liveborn males, regardless of ethnicity. Since gene defect is X-linked recessive, symptoms usually appear in males (XY-chromosomes) at 1 to 6 years old. Females (XX-chromosomes) are carriers of the gene for this disorder and rarely develop symptoms. Treatment is aimed at control of symptoms to maximize the quality of life. Mild activity is encouraged. Inactivity (such as bed rest) can worsen the muscle disease. Excess activity can speed up the disease. Physical therapy may be helpful to maintain muscle strength and function. Orthopedic appliances such as braces and wheelchairs may improve mobility and the ability for self-care. DMD results in rapidly progressive disability. DMD is the most common, severe, and progressive muscular dystrophy to affect children. It is also one of the most common, inherited, and lethal pediatric neuromuscular disease. Patients are lacking dystrophin from early fetal life, and show evidence of myofiber membrane instability from birth along with elevated serum creatine kinase and degeneration/regeneration on muscle biopsy; however, weakness is not observed until 3-4 years of age. This pediatric disorder exhibits an inheritance pattern that is X-linked recessive, so boys tend to be predominantly afflicted whereas girls tend to be carriers. The natural history for boys with DMD is difficulty walking by 3 years of age, wheel-chair bound by 12 years of age, and usually death succumbing to cardiorespiratory failure by 20 years of age.

There is no specific therapy except supportive treatments such as leg braces, tendon releases, and respiratory assistive devices. A pharmacologic therapy has been established which includes 0.75 mg/kg prednisone in intermittent regimens. The therapy has been shown to prolong independent ambulation but has significant side-effects (e.g. weight-gain, osteopenia, behavioral and endocrinologic issues). Moreover, oral steroids have not been shown to prolong the life expectancy of young men with DMD. By age 10, braces may be required for walking, and by age 12, most patients are confined to a wheelchair. Muscular weakness and skeletal deformities contribute to frequent breathing disorders. Cardiomyopathy occurs in almost all cases. Intellectual impairment is common but is not inevitable and does not worsen as the disorder progresses. Death usually occurs by age 15, typically from respiratory (lung) disorders.

Advances in research have greatly enhanced specific diagnostic testing for identifying patients and carriers, however conventional treatments for patients remain unchanged. To date, there is no specific therapy or treatment for DMD that can extend the expected lifespan of boys afflicted with DMD.

The underlying genetic basis for DMD has been defined as a loss-of-function mutation in the dystrophin gene, which encodes a 427-kD protein thought to have a cytoskeletal, membrane-linking, and other functional roles. A mouse model of the DMD disease (mdx) also lacks the same dystrophin protein, but the mice are surprisingly not crippled. In fact, these mice are reproductively viable, live near full lifespans, and can run up to 8 km/day (vs 10 km/day in wild). The mdx mouse has skeletal muscle histology characterized by minimal fibrosis and over 75% of myofibers with central myonuclei, which are two prominent features considered differential to human DMD. These skeletal muscle tissue differences reflect desirable adaptations and are presumably governed by transcriptional activity which can be globally investigated with new genomic and bioinformatics technology. Such large-scale genomic screening has a distinct advantage of being remarkably unbiased, a perspective that favors gene discovery. In other words, mdx mice can secondarily compensate to a remarkable degree in the absence of dystrophin protein, unlike humans with DMD or Golden Retriever Muscular Dystrophy dogs (GRMD). Although there are scores of laboratories working to describe the function(s) of the dystrophin protein, there is a paucity of information describing how the absence of dystrophin protein manifests differentially into a benign phenotype in mice, but not dystrophin-deficient dogs or humans.

The study of model organisms with knockouts or related genetic manipulations is a central theme of many efforts to understand inherited diseases in humans. However, it is not always the case that the phenotype produced by the genetic manipulation of the model organism corresponds well to the human phenotype. Diseases for which mouse knockouts failed to produce human-like phenotypes include Tay-Sachs, Fabry disease, Lesch-Nyhan syndrome, and Duchenne Muscular Dystrophy (DMD). Although such failures preclude the use of the model system to study etiology directly, differences in susceptibility can themselves be used to interrogate the effects of genetic background on disease, potentially illuminating important protective or compensatory factors that could play a role in the development of novel therapy.

Many laboratories have attempted to identify differential adaptations that underlie selective species and selective muscle vulnerability in dystrophin-deficient animal models. Recently, five different expression profiling studies have reported on mature hindlimb skeletal muscle differences between mdx mice and distrophin intact mice on the same C57 genetic background. No clear pattern emerged from the conclusions of these papers. Individually, the five selected mdx expression profile studies generated data but made limited or no general summaries from their expansive data, totaling 642 differential candidate genes. In the mdx mouse there are likely to be many differential genes, some that could have important compensatory roles, others with irrelevant secondary effects, and a proportion that could be sheer artifacts of these new technologies.

In a paper by Yasuda et al. (Nature 436:1025-1029, 2005) there was disclosed the positive effect on cardiac myocytes in isolated cells and in-total preparations in various conditions to monitor membrane integrity (e.g. intracellular calcium measures and contractile force under dobutamine stress tests). The poloxamer 188 compound employed showed beneficial effects in single timepoint studies from mdx mouse hearts. However, to date, there have been no published timecourse studies with skeletal muscle in the mdx mouse. It would therefore be beneficial to develop a treatment of skeletal muscle to overcome the problems disclosed above.

### SUMMARY OF THE INVENTION

According to the present invention, there is provided a molecular sealant comprising a therapeutically effective amount of a poloxamer capable of sealing a cell or tissue from leaks. A method of treating tissue leakages by applying the above sealant to the tissue or cell in need of treatment. A composition for treating cellular leakages comprising a poloxamer capable of sealing a cell or tissue from leaks in a pharmaceutically acceptable carrier.

### BRIEF DESCRIPTION OF THE DRAWINGS

FIGS. 1A-I are photographs (FIGS. 1A-H) and a graph (FIG. 1I) showing the analysis of whole-calf anti-albumin treatment;

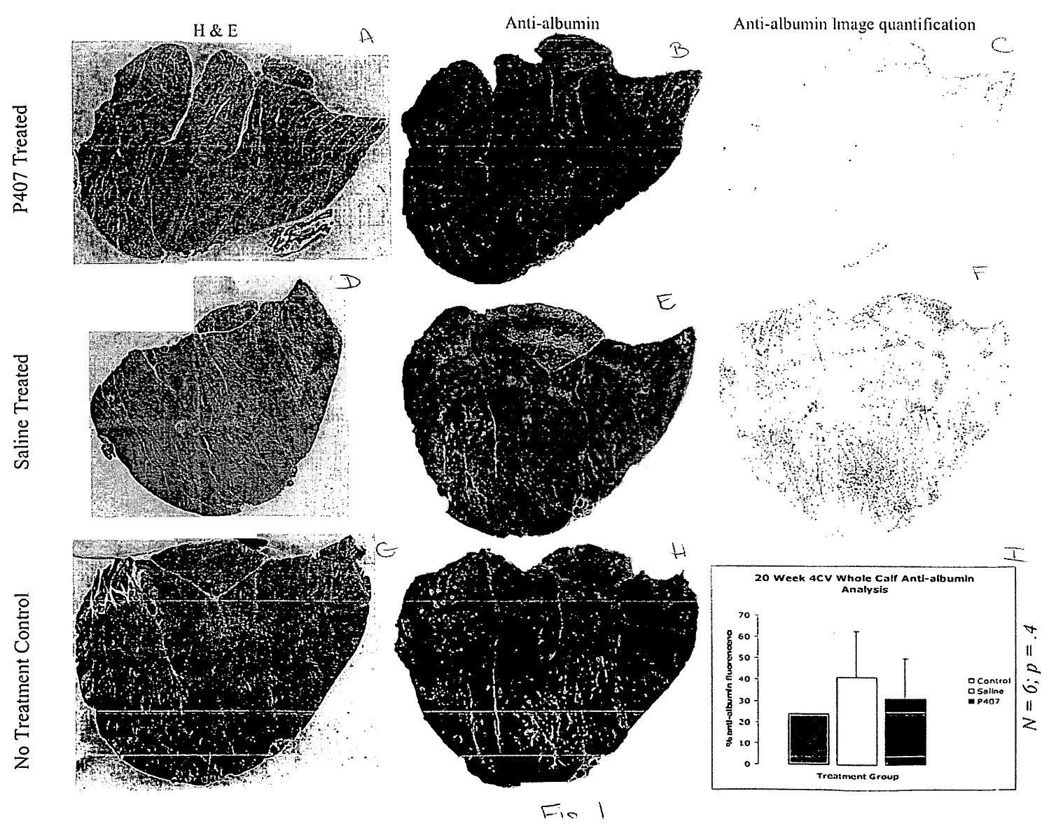

FIGS. 2A-D are photographs (FIGS. 1A-C) and a graph (FIG. 1D) showing the analysis of rectus anti-albumin treatment;

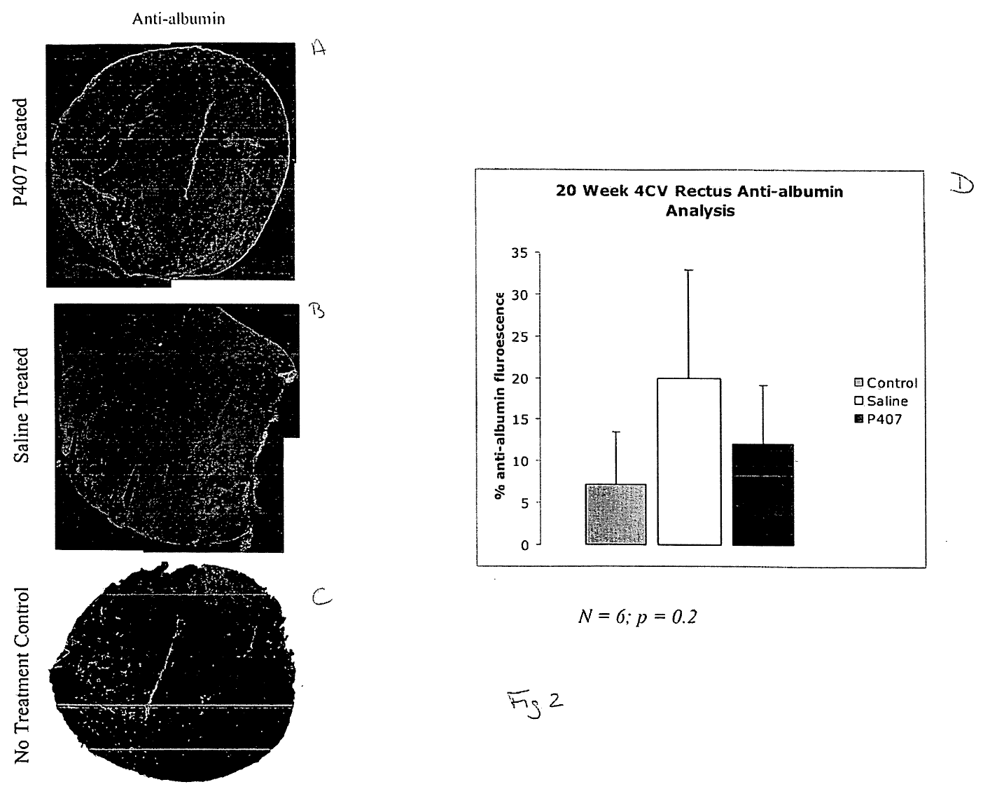

FIGS. 3A-D are photographs (FIGS. 3A-C) and a graph (FIG. 3D) showing the analysis of soleus fiber anti-albumin treatment;

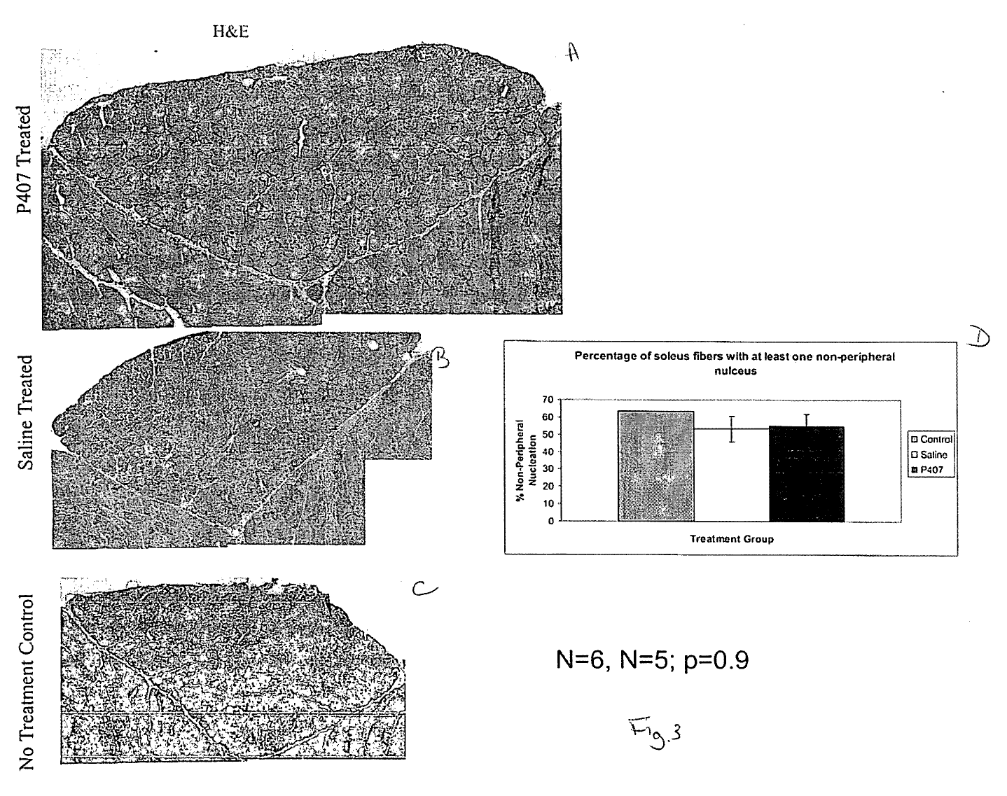

FIGS. 4A-D are photographs (FIGS. 4A-C) and a graph (FIG. 4D) showing the analysis of rectus femoris fiber anti-albumin treatment;

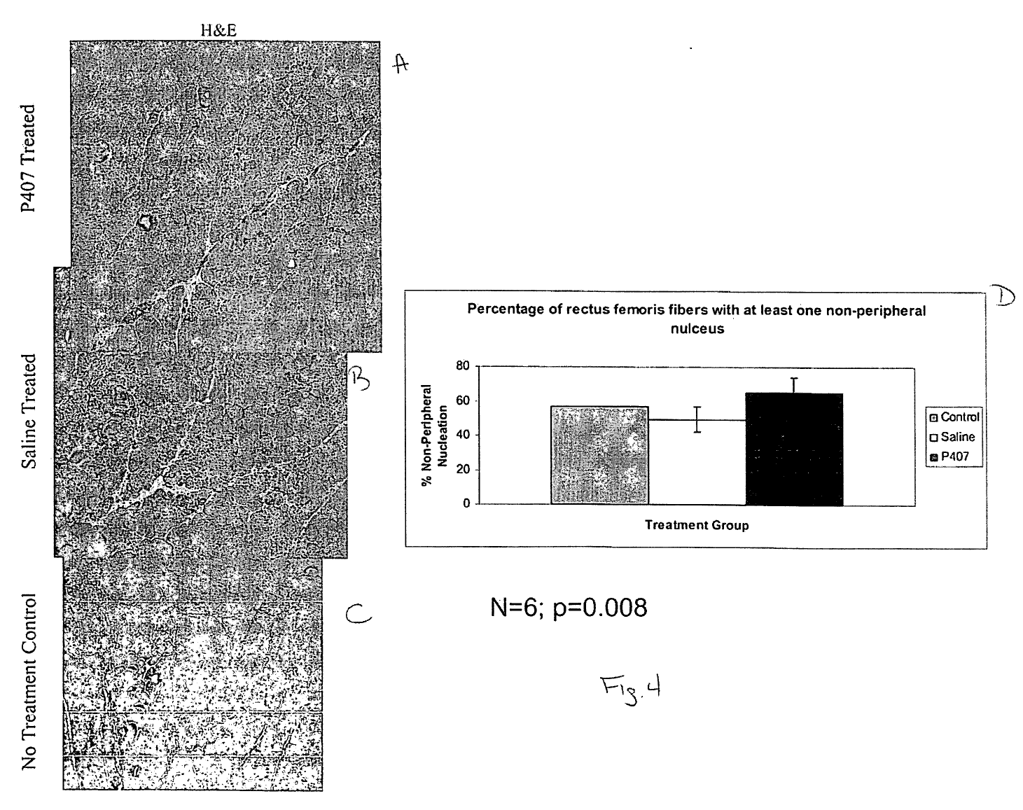

FIGS. 5A-D are photographs (FIGS. 5A-C) and a graph (FIG. 5D) showing the analysis of whole calf EmbMyHc;

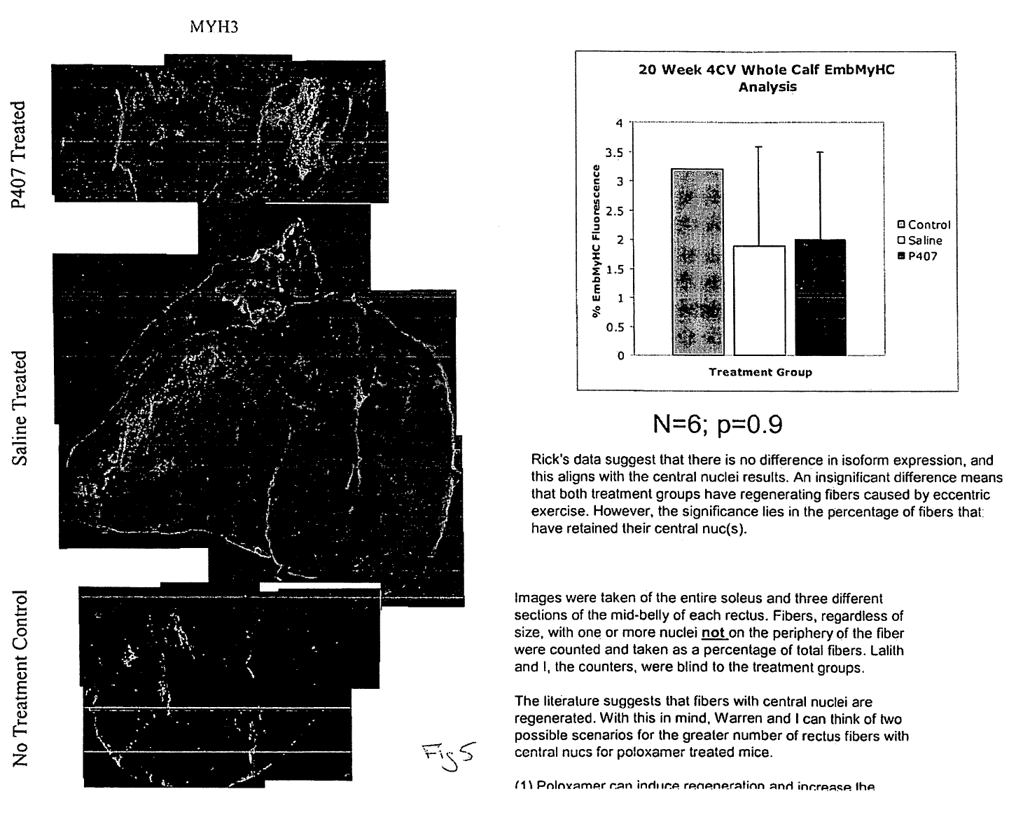

FIGS. 6A-D are photographs (FIGS. 6A-C) and a graph (FIG. 6D) showing the analysis of rectus EmbMyHc;

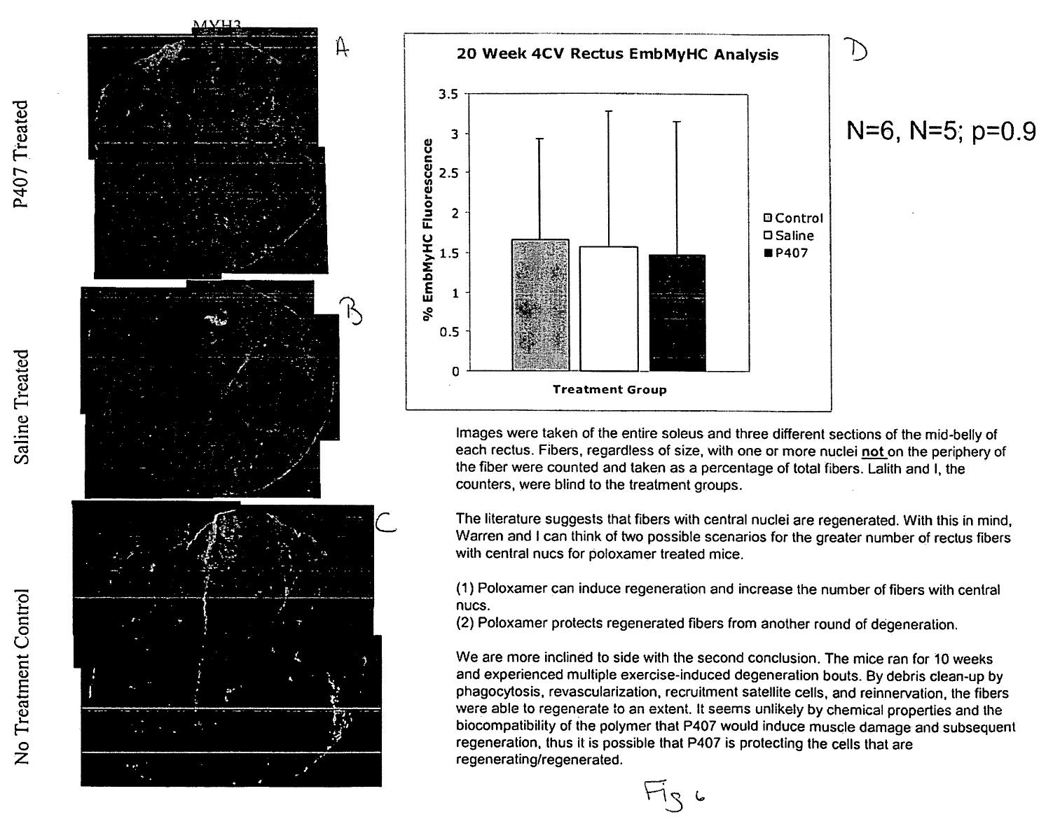

FIG. 7 is graph depicting the results for the poloxamer study;

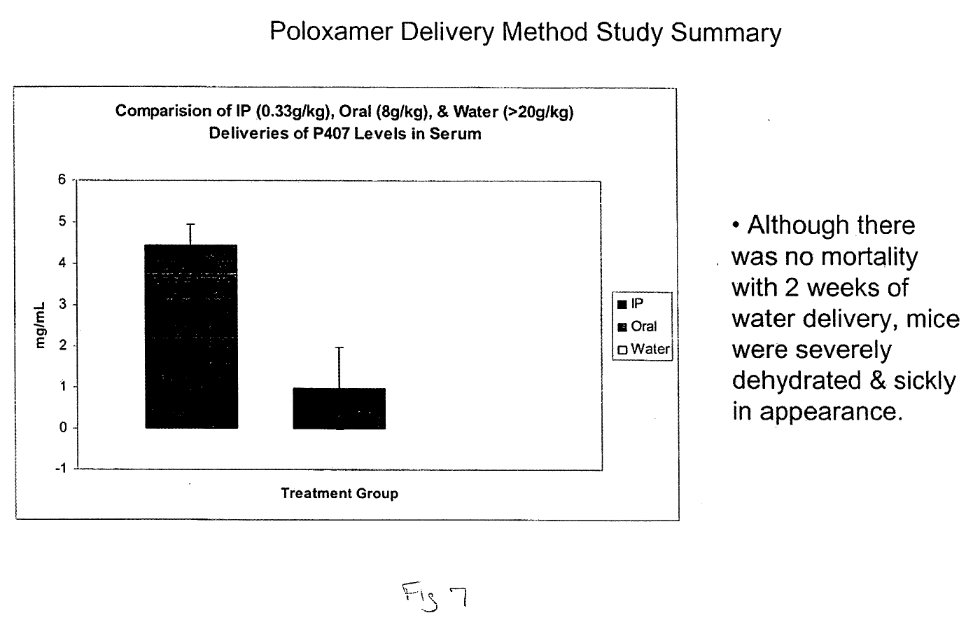

FIGS. 8A-K are photographs showing gastrocnemius muscle illuminated by P407 tagged with FITC; controls and variable displayed with equal exposure of 100 ms and auto-normalized, wherein the P407/FITC localized in heart and skeletal muscle;

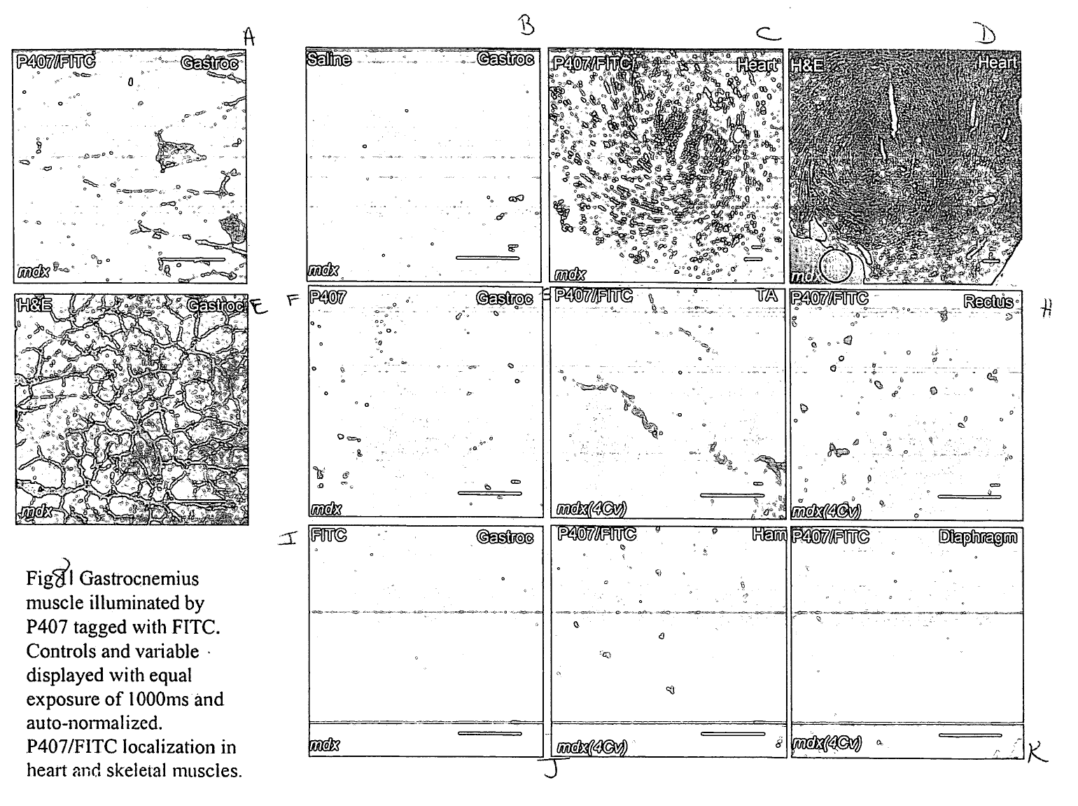

FIGS. 9A-D are photographs (FIGS. 9A-C) and a graph (FIG. 9D) showing the timelapse of treatment, wherein days 1-3 were taken with 20 ms exposure and day 4 was taken at 100 ms exposure; all images were taken at the 40× objective and P407 serum concentrations were measured with the modified cobalt thiocyanate assay;

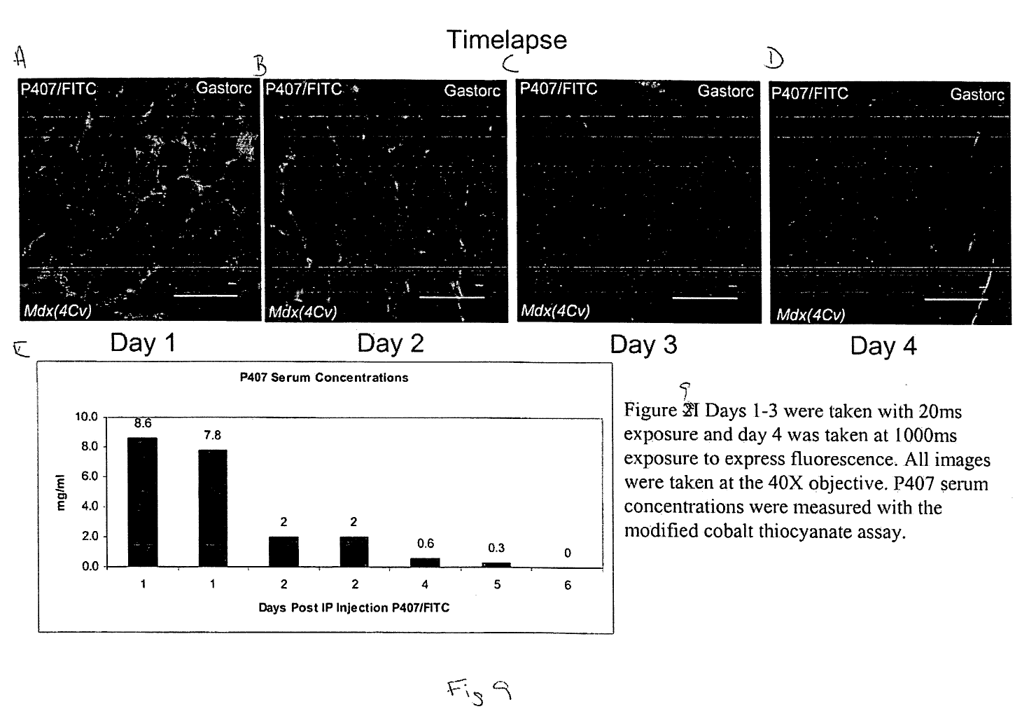

FIG. 10 is a graph showing the effect of three months of treatment with an i.p. dosage of 0.8 g/kg every third day;

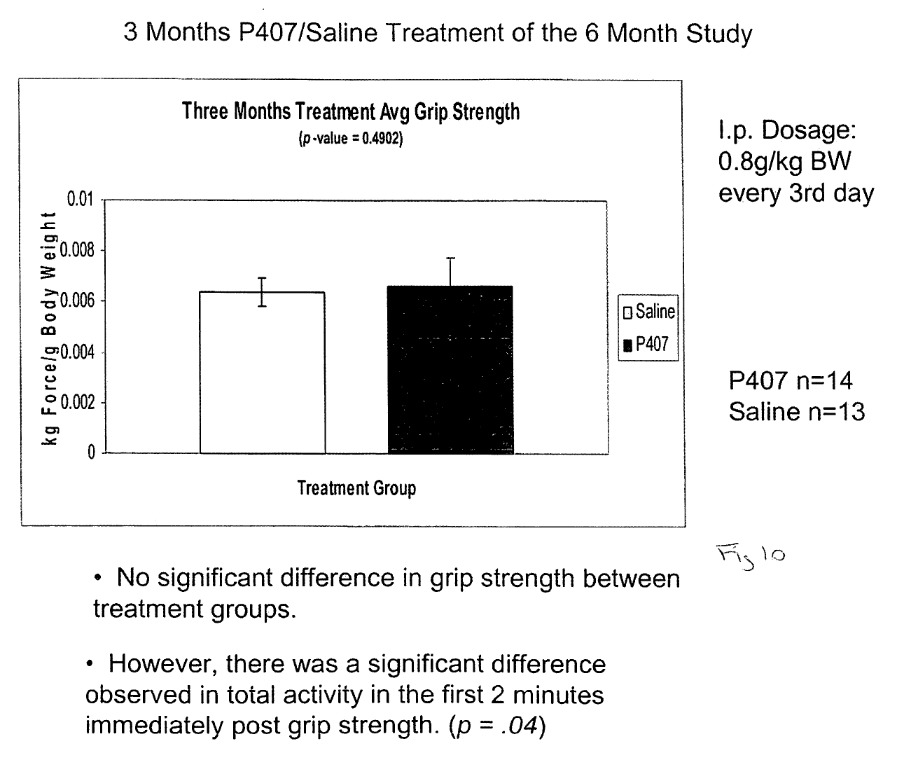

FIGS. 11A and 11B are graphs showing the effect of treatment on activity;

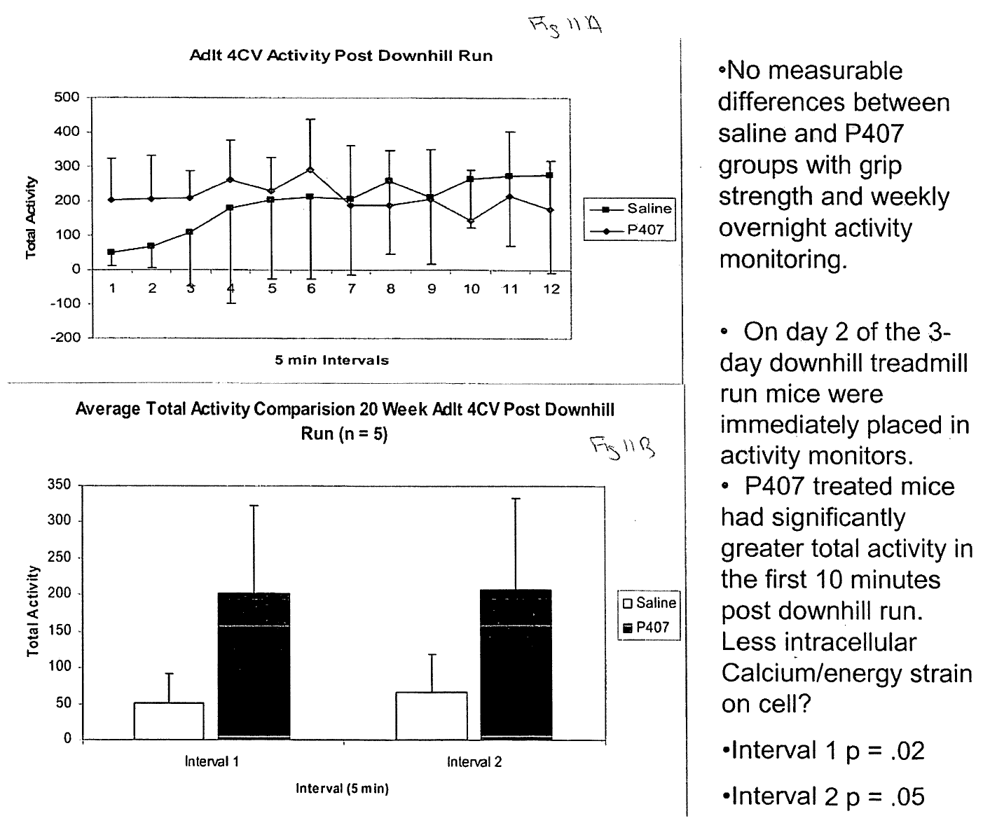

FIGS. 12A-K are photographs (FIGS. 12A-1) and graphs (FIGS. 12J-K) showing the results of the treatments; and

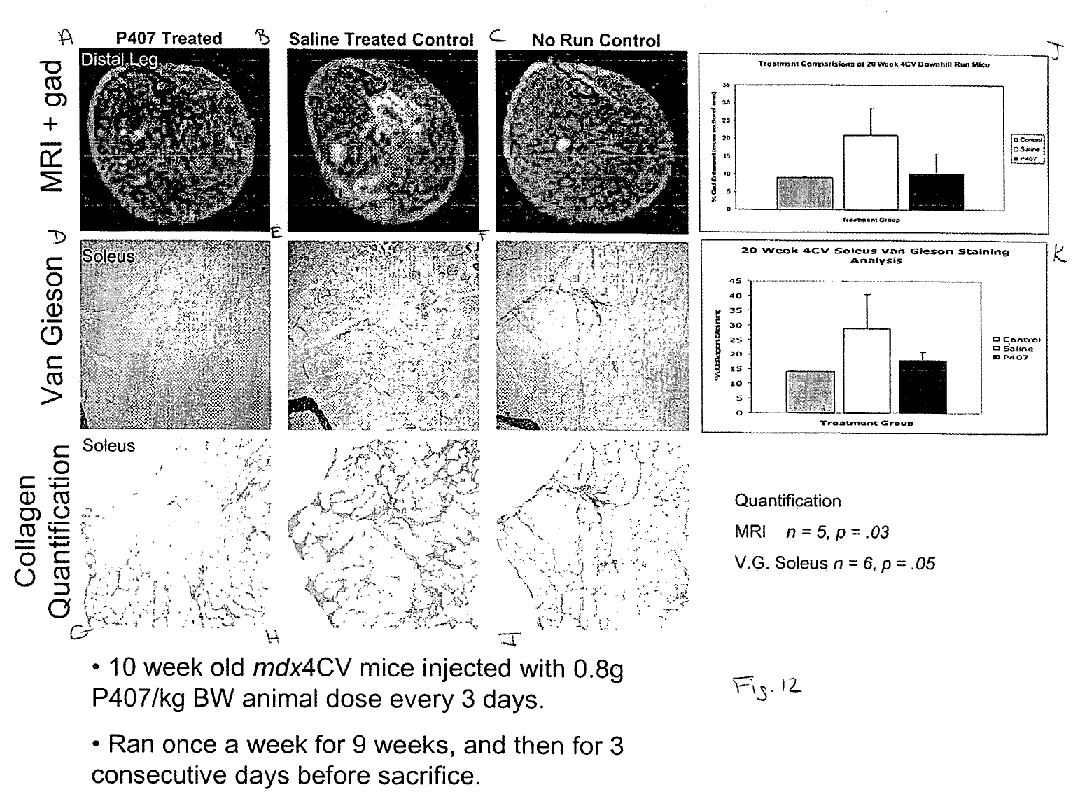

FIGS. 13A-D are photographs (FIGS. 13A-C) and a graph (FIG. 13D) showing the analysis of rectus Van Gieson staining.

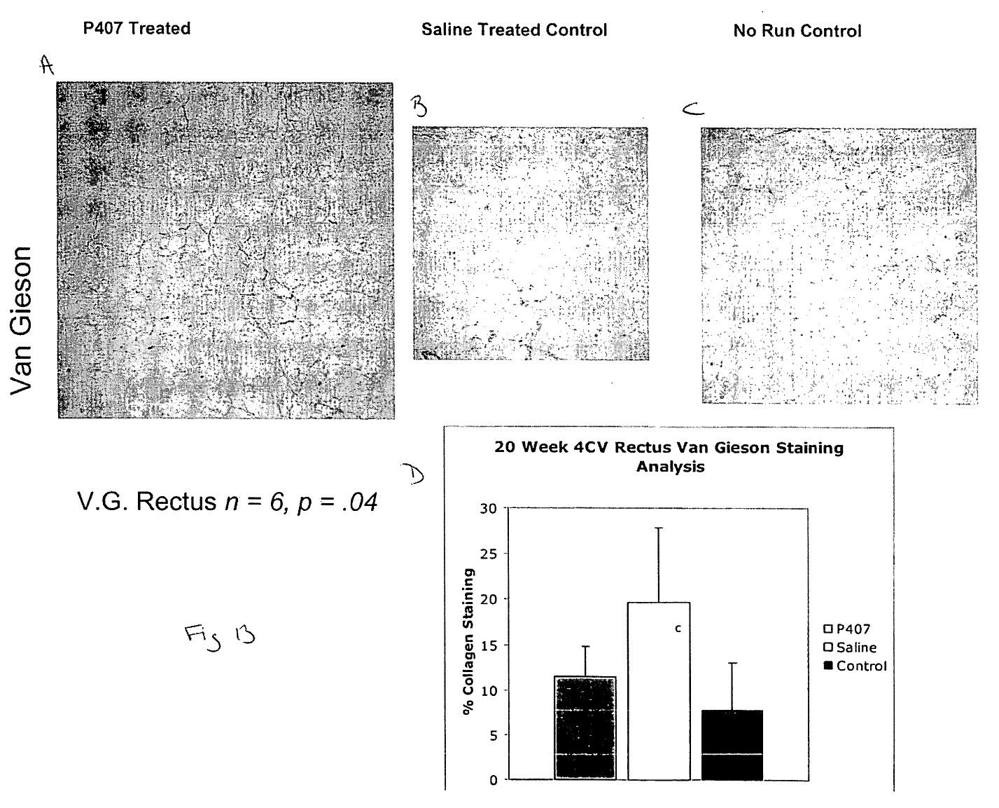

### DESCRIPTION OF THE INVENTION

The present invention provides a membrane sealant or “molecular band-aid.” The sealant is used to patch tissue, most preferably muscle tissue to prevent and correct muscle membrane leaks.

The term “sealant” as used herein is intended to include, but is not limited to a compound or composition that is able to prevent the leakage of a necessary cellular component. The sealant compound is biologically safe and can be injected, ingested, or delivered subcutaneously. The sealant is preferably a poloxamer, but can be another compound that is biologically acceptable and can perform the functions disclosed herein. Most preferably, the compound is poloxamer 407 or functional equivalents thereof known to those of skill in the art.

The sealant of the present invention can be used to treat cellular leakages. Preferably, the sealant is used to treat muscular cell leakages. In the preferred embodiment, the sealant is used to treat skeletal muscular cell leakages. Such cells are different than other muscle cells in that compounds that can be used to treat other types of muscle cells are not always useful in treating skeletal muscle cells. In contradistinction, as the skeletal muscle cells are more difficult to treat, the compounds used to treat skeletal muscle are often useful in treating other types of muscle cells.

A compound called poloxamer 407 can serve as a “molecular band-aid” to patch muscle membrane leaks and improve skeletal muscle integrity and function. Poloxamer 407 is a safe compound which, depending on grade, can vary from a liquid to a solid form. Poloxamer 407 is found in daily household commodities such as toothpaste, alka-seltzer, mouthwashes, children's cavity rinses, suppositories and also used as the solid coating of many pharmaceutical pills and tablets. In fact, poloxamer compounds are being tested in two human Phase I trials as a carrier for genetic vectors in cancers and alone in sickle cell disease for treating vaso-occlusive chest syndrome. In the present invention the compound is used to “plug” into membranes and most importantly improve skeletal muscle function in animals.

In boys with human Duchenne muscular dystrophy (DMD), dystrophin protein deficiency causes skeletal muscle degeneration, resulting in wheelchair dependence by age 12 and fatal cardio-respiratory disorders by the mid-20 s. This skeletal muscle defect is due to excessive membrane “leaks” or permeability with disrupted dystrophin-dystroglycan complexes, which leads to both inappropriate efflux of intracellular metabolic enzymes (e.g. creatine kinase CK) and excessive influx of extracellular components (e.g. increased calcium and albumin within muscle fibers). A physiopathological model of human DMD is the mdx mouse. The mouse similarly lacks dystrophin protein and, consequently, possesses similar dystrophin-deficient “leaky” muscle cell membranes, thus emulating the skeletal muscle tissue defects in human DMD. Poloxamer 407 represents a new therapeutic treatment for dystrophic skeletal muscle.

Poloxamer is a family of amphipathic block copolymers, consisting of a central hydrophobic block flanked by two hydrophilic-hydroxyl wings. Specifically, P407 is a copolymer consisting of a hydrophobic core of polypropylene oxide edged by two hydrophilic wings of polyethylene oxide (Fussnegger 2002). Hydrophobic phospholipid interactions via the Poly(phenylene oxide) (PPO) core and the hydrophilic phospholipids interactions via the polyethylene oxide (PEO) wings theoretically allow poloxamer to sterically reduce membrane perforations. Due to its low toxicity, poloxamer 407 is a viable candidate to strengthen or patch the dystrophic skeletal muscle cells. Poloxamer 407's structure enables the composition to “plug” the membrane holes as it partially mimics the phospholipid structure. A study by Yasuda et al. with poloxamer 188 in single cardiomyocytes and in heart preparations from mdx mice showed beneficial effects in vitro. Poloxamer 188 administration in vitro appeared to inhibit acute mdx cardiac failure during a dobutamine-induced stress experiment. Poloxamer 188 is too small to help the larger holes in skeletal muscle, whereas poloxamer 407 serves as a bigger and more effective membrane patch.

Poloxamer 407 was chosen as the compound of interest because it is larger (12,600 MW) than Poloxamer 188 (11,400 MW) and it has slightly different properties e.g. less hydrophobic and can be tagged with markers such as DTAF (495 daltons). Based on this, in conjunction with poloxamer 407 pilot data from seven day studies in mdx mice, it was determined that poloxamer 407 is a safe, membrane integrating compound which can help fortify leaky mdx skeletal muscle membranes. As such, poloxamer 407 is a biologically safe compound to give animals. Poloxamer 407 is a compound that has unique chemical properties to integrate into lipid membrane bilayers in skeletal muscle membranes of mdx mice. Flourescently tagged poloxamer can be synthesized and administered in an effort to determine localization in skeletal muscle membranes of primary muscle cell cultures under confocal microscopy and with fluorescent microscopy of tissue sections from in vivo poloxamer treated mdx mice. In fact, flourescently tagged P407 has been formed and its membrane localization has been established.

The treatment of the present invention can result in decreased blood levels of serum CK, improved skeletal muscle histology (H&E staining), decreased muscle fiber levels of intracellular albumin and decreased Evans blue uptake, reduced fibrosis by hydroxyproline assays, and mdx mice treated with poloxamer 407 will have better running and recovery performance in voluntary running mouse wheels, grip strength and downhill treadmill running than untreated mdx.

The composition of the present invention can be used as a treatment of Duchenne muscular dystrophy that can extend beyond the marginal benefits of corticosteroids and may synergize with developing cell/stem and gene-based replacement strategies. The present invention is focused on developing bridging-therapies that may not necessarily be ultimate genetic replacement cures but will be important disease modifiers to slow the pathogenesis of dystrophin-deficiency disease and buy time for boys with DMD.

The invention is further described in detail by reference to the following experimental examples. These examples are provided for the purpose of illustration only, and are not intended to be limiting unless otherwise specified.

Thus, the invention should in no way be construed as being limited to the following examples, but rather, should be construed to encompass any and all variations which become evident as a result of the teaching provided herein.

### EXAMPLES

### Methods

General methods in molecular biology: Standard molecular biology techniques known in the art and not specifically described are generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

General methods in immunology: Standard methods in immunology known in the art and not specifically described are generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

### Delivery of Therapeutics (Compound):

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses may be single doses or multiple doses over a period of several days, but single doses are preferred.

The doses may be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered can vary for the patient being treated and will vary from about 100 ng/kg of body weight to 100 mg/kg of body weight per day and preferably can be from 1 mg/kg of body weight to 10 mg/kg of body weight per day.

### Example 1

### Materials & Methods

### Fluorescent Labeling and Purification of P407

P407 was fluorescently conjugated with 5-(4,6-dichlorotriazinyl)aminofluorescein (5-DTAF) by the technique described by Langmuir. (Frey S L et al. 2007 and Maskarinec S A et al. 2002) In brief, a stock solution of P407 6% w/v was prepared by dissolving P407 in phosphate buffered saline (PBS) at pH=10.5. A stock solution of 100 g/mL 5-DTAF was prepared by dissolving in dimethyl sulfoxide (DMSO). The 5-DTAF solution was added to the P407 solution in a 3:1 molar ratio. The reaction was allowed to proceed in a rotating hybridization oven at 30° C. for six hours at a low revolution speed. Upon completion, the reaction yielded a fluorescently labeled P407 and excess 5-DTAF. Column chromatography with Bio-Gel P-4 Gel Medium 90-180μ (Bio-Rad, Hercules, Calif.) was used to separate the desired tagged P407 from the lower molecular weight (˜495 Da), excess 5-DTAF. The labeled P407 fractions were then further purified using Centriplus YM-10 10 kDa molecular weight cut-off centrifugal filtration devices (Millipore, Bedford, Mass.) at 5000×g for 60 minutes at 20° C. The separation and purification steps were carried in the dark to avoid possible photobleaching of 5-DTAF. In preparation for i.p. administration, concentrations of 5-DTAF and P407 were measured and labeling reaction yields were recorded for the purified fractions, which were then stored in the dark (Langmuir 2001).

### Serum Modified Cobalt-Thiocyanate P407 Concentration Assay

The concentration of P407 in serum samples post i.p. administration was measured according to the reagents and modified protocol of Langmuir et al. P407 standards were established in the concentration range of 0.3-20 mg/ml. A mixture of 10 μl serum or P407 standard, 10 μl cobalt-thiocyante (Sigma-Aldrich St. Louis, Mo.) reagent, 10 μl ethyl acetate (Sigma-Aldrich), and 4 μl absolute ethanol (Pharmco-Aaper Alcohol and Chemical Company Shelbyville, Ky.) was gently spun at 12,000×g for ten minutes in Hermle Z300K centrifuge at 4° C. (Labnet Edison, N.J.). Following the spin that yielded a dark blue pellet consisting of a cobalt-thiocyante and P407 complex, the pellet was successively washed approximately 4-6 times with 500 μl of ethyl acetate and spun at 12,000×g for two minutes at 4° C. until a clear supernatant was observed. After the last wash, the supernatant was removed and pellet was suspended in 200 μl acetone (Sigma-Aldrich). To dissolve the pellet, samples were rigorously vortexed and placed in a heating block at 60° C. for 30 minutes. The absorbance was measured at 623 nm using a DU-640 Spectrophotometer (Beckman Corona, Calif.) and concentrations were determined based on calibration standards.

In vivo P407 vs Saline Treatment

In Vivo Dosaging

Intraperitoneal Injections Preparations
(0.8 g P407/kg BW every 4^{th} day) P407 10% w/v in 0.9% NaCl Saline vs 0.9% NaCl Saline

The mice are given 0.33 g/kg/day of a 10% w/v solution. This dose is also used for the pharmacokinetics studies. However, when striving for a therapeutic effect, this dose did not suffice. It was noted that administration of 1.0 g/kg/day of a 10% w/v solution for 4 days demonstrated distinct splenomegaly when compared to control animals. A significant reduction in body weight and significant decrease in the percent of lymphocytes, red blood cells, hemoglobin, and percent hematocrit were noted. A side-effect of hypercholesterolemia is also known to occur with P407 administration at doses greater than approximately 0.6 g/kg/day. The present study used the 0.8 g/kg/ every 4 days and the beneficial results are more significant than the more dilute doses. The main reason for the increased dosage was to try and get higher serum levels to try and bring out more of a noticeable (function) effect, and that we were under the LD50 with 0.8 g/kg dose (MSDS).

### Experimental Design

### P407 Concentration Time-Lapse Study

Eight adult age-matched mdx4cv mice were injected with 29 μl of labeled P407 per gram animal of an 8.82 μg P407 per μl phosphate buffered saline solution to achieve 0.33 g P407 per kg animal BW dose. 24, 48, 96, 120, 144 hours post i.p. injection tissue and serum was collected and harvested. The intended 72-hour analysis mouse died before tissue collection was possible of unknown cause.

### Fluorescence

### P407 Serum Concentration

Blood was collected via retro-orbital eye bleed prior to sac on the days indicated. Serum was separated from whole blood at 5000 rpm for 10 minutes at 4° C. The modified co-thio assay was used to determine P407 serum concentrations.

### Delivery Methods Study

### Delivery Mediums

Age-matched male mdx4cv were intraperitoneally given 0.33 g P407/kg BW 10% P407/Saline IP injection for 5 days, 25 g P407/kg BW 10% P407/Water for 2 weeks, and 2 g P407 in 2.5 g Nutri-Cal/kg BW (Fed everyday for 5 days).

P407 Solution Preparation for i.p. Administration

P407 (BASF Florham Park, N.J.) was dissolved in 0.9% NaCl sterile saline to yield a 10 wt % P407 solution, which was determined to be the most bioavailable and least viscous for i.p. injection. The P407 powder was allowed to dissolve into the saline solution overnight with mild agitation at 60° C. in a hybridization oven.

### Example 2

There is shown that in the mdx 4CV mouse the block copolymer poloxamer 407 localizes to the membranes, thus showing modulation of dystrophic skeletal muscle integrity based on counts of central nuclei, embryonic MHC, Evans Blue and anti-albumin markers. There is shown improved spontaneous activity immediately post eccentric exercise and grip strength. Six months of intraperiotoneal administration of poloxamer 407 protects against fibrosis with Van Gieson stains and muscle degeneration/regeneration markers on muscle MRI with intraveneous gadolinium enhancement.

To investigate functional restoration of dystrophic skeletal muscle, a placebo-control study was performed on DMD^{mdx}, mdx4cv treated with intraperitoneal (i.p.) injections of P407 and saline solution.

A previous study using another poloxamer, poloxamer 188, at the single cardiac myocyte level corrected the detrimental effects of calcium overload in vitro. The administration of poloxamer 188 in vivo inhibited acute cardiac failure during a dobutamine-induced stress experiments (Metzger 2005). However, a different study showed that poloxamer 188 fails to prevent exercise-induced membrane degradation of the mdx skeletal muscle sarcolemma (Quinlan 2006).

The chemical properties of P407, poly(ethylene oxide)₁₀₁-poly(propylene oxide)₅₆-poly(ethylene oxide)₁₀₁ (molecular mass ˜12.6 kDa), are better-suited for spanning the larger tears due to increased mechanical stress in the sarcolemma of dystrophic skeletal muscle rather than P188, poly(ethylene oxide)-₈₀-poly(propylene oxide)₂₇-poly(ethylene oxide)-₈₀ (molecular mass ˜8.4 kDa). P407 localizes to skeletal muscle membranes and improves some surrogate markers of DMD.

### Materials & Methods

Adult mdx4cv breeding pairs were obtained from Dr. Jeffrey Chamberlain at Washington University in St. Louis, Mo. All mouse protocols were approved by the Institutional Animal Care and Use Committee of the University of Colorado Denver Health Science Center. Protocol number 67602103(09)C

### Histology/Fluorescence Microscopy

### Tissue Handling

Mice anesthetized by intraperitoneal injection of 60 mg/kg ketamine and 10 mg/kg xylazine and cervical dislocations were performed prior to tissue harvesting. Hindlimb muscles were extracted, mounted in Optimal Cutting Temperature (Tissue-Tek Torrance, Calif.) medium, and flash-frozen in liquid nitrogen cooled to −160° C. isopentane. Tissue was cut in 8 μm serial sections using a Microm HM 505 E cryostat (Global Medical Instrumentation, Inc Ramsey, Minn.). Sections were mounted and coverslipped with anti-fading aqueous mounting medium on Superfrost Plus Gold slides from Fischer Scientific.

### Hematoxylin and Eosin (H&E)

Tissue morphology including central nuclei quantification in the rectus femoris and soleus muscles was analyzed using H&E staining. Tissues sections were exposed to Harris modified hematoxylin with acetic acid (mercury-free) (Fisher Scientific Fair Lawn, N.J.) for one minute, rinsed with distilled water, exposed to Eosin Y solution (intensified) for two minutes, rinsed with tap, and dehydrated with a series of increasing concentrations of ethyl alcohol ending with xylenes. The sections were then coverslipped using Permount (Fisher Scientific Fair Lawn, N.J.) histological mounting medium.

### Van Gieson

Intramuscular collagen was stained using Van Gieson solution (Sigma-Aldrich St. Louis, Mo. Ref. HT254-250 ml) in the rectus femoris and soleus muscles. Tissue sections were exposed to Van Gieson solution for five 30 minutes and dehydrated with a series of increasing concentrations of ethyl alcohol ending with xylenes (Cohn 2007). The sections were then coverslipped using Permount (Fisher Scientific Fair Lawn, N.J.) histological mounting medium.

### Evans Blue Dye (EBD)

Membrane disruption in the rectus femoris and soleus muscles as a result of eccentric exercise was measured using EBD. Labeling was achieved by intraperitoneal injection of 50 μl per gram body weight of a 2% EBD saline solution 4 hours prior to sacrifice. (Ikeda 1988 and Quinlan 2006)

### Immunofluorescence

### Anti-Albumin

Collected tissues were sectioned for fluorescence immunostaining. Serial sections of rectus femoris and soleus muscles were mounted on slides and prepared for anti-albumin staining by blocking with 10% goat serum/PBS for 30 minutes at RT. The sections were then probed with polyclonal goat anti-mouse albumin antibody fluorescein isothiocyanate (FITC) (Bethyl Laboratories Montgomery, Tex.) cross absorbed (1:200 in blocking solution) for 90 min at RT in the dark. The sections were washed 2×2 min in PBS, stained with Hoechst stain solution for three minutes (Sigma-Aldrich St. Louis, Mo.), washed with PBS for two minutes, and coverslipped, all at RT (Straub 2007).

### Embryonic Myosin (MYH3)

Serial sections of rectus femoris and soleus muscle were also probed for MYH3 using a Vector Mouse on Mouse (M.O.M.) kit (Vector Labs Burlingame, Calif.) to identify single regenerating fibers. Tissue sections were blocked with steptavidin and biotin according to Vector labs kit. The sections were then incubated with mouse monoclonal IgG1 MYH3 (1:10) (purchased from the Developmental Studies Hybridoma Bank-University of Iowa) (Blau) for 30 minutes. Following the primary antibody, the sections were incubated with secondary biotinylated anti-mouse IgG for 10 minutes and incubated with tertiary Alexa 488 anti-mouse IgG steptavidin (1:400) for 45 minutes in the dark. All steps were carried out at RT according to the immunofluorescence protocol for M.O.M. kit (Matecki 2004).

### Imaging

Collagen quantification was performed on Van Gieson stained mid-belly regions of the rectus femoris and soleus muscles. With EBD, anti-albumin, and MYH3 staining, whole rectus femoris and calf sections were analyzed. Images were acquired by investigators blinded to treatment cohorts with IPLab v4.04 (BD Biosciences Rockville, Md.) software using Qlmaging Retiga 4000R Fast 1384 camera with X5 and X10 objectives on a DM4000V microscope (Leica Inc.).

All images were acquired under the same parameters of exposure time, contrast, and normalization.

### Quantification

Sections were analyzed using Adobe Photoshop CS3 software (Version 10.0). Color saturation and hue were adjusted to enhance the contrast of the pathologic areas, which were isolated using the wand tool. The contrast tolerance was set at 25 for Van Gieson stains, 44 for EBD, anti-albumin, and MYH3, and 20 for MRI analysis. These isolated regions were then compared to the original images to verify that the representative areas of interest were captured in their entirety. Negative control sections also served as minimums of selection criteria. A numerical value of pixel saturation for single colors was displayed and used as a basement for all experimental sections. Subsequently, quantification was preformed using the record measurement feature of the Adobe software. This information was then exported to Microsoft Excel and decoded for statistical analysis to determine amount dystrophic related pathology between the P407 and saline treatment groups.

### Magnetic Resonance Imaging (MRI)

Mice were anesthetized by intraperitoneal injection of 60 mg/kg ketamine and 10 mg/kg xylazine and placed on the heating pad. A tail vein catheter was started on each mouse using winged infusion set with a 27 gauge needle (Becton Dickinson Cat# 63119-928) immediately prior the imaging session. In our mouse model, 0.1 mmol/kg gadodiamide (OMNISCAN® 287 mg/ml), previously diluted in bacteriostatic 0.9% sodium chloride as 1:10 (v/v), was administered intravenously in the tail vein of the mouse during fast T1-MRI scan series (DCE-MRI, see below). The dilution was performed prior to each experiment and the total volume of tail vein injection did not exceed 200 μl, with 120 μl heparin-containing flush-solution injected prior to 80 μl gadolinium solution to avoid pre-enhancement. The animals were fixed in a mouse animal bed and inserted into a Bruker volume coil (36 mm diameter), tuned to the ¹H frequency of 200 MHz, which was used for radiofrequency (RF) transmission and receiving. The proton-density, T1- and T2-weighted MR images were performed at a Bruker PharmaScan animal scanner (Bruker Medical, Billerica, Mass.) at 4.7 Tesla equipped with ParaVision software (version 3.2).

### Multi Slice Multi Echo (MSME)

T1-weighted scans were performed prior to Gd injection (first set) and an additional set was carried out 3 min post injection. The following parameters: field of view (FOV) 3.2 cm, slice thickness 1 mm with no gap between slices, with 16 axial slices, matrix size 256×256, TR=720 ms, TE=11 ms, number of averages 2, total scan time 6 min 8 sec (McClure 2007, manuscript in review).

### MRI Parameters

The fast spin echo RARE (rapid acquisition with relaxation enhancement) proton density-(PD) and T2-weighted scans were done for comparison. PD parameters were: FOV 3.2 cm, slice thickness 1 mm no gap, with 16 slices, matrix size 256×256, TR=2000 ms, TE=31.9 ms, number of averages 4, total scan time 2 minutes 8 seconds). T2 parameters were as follows: FOV 3.2 cm, slice thickness 1 mm no gap, with 16 slices, matrix size 256×256, TR=5000 ms, TE=110 ms, number of averages 2, total scan time 5 minutes 19 seconds).

### MRI Quantification

Gadolinium enhancement was measured using ParaVision software. Investigators were blinded to the treatment groups and analysis was preformed by taking a percentage of the determined total area of T-2 signal with respect to the area of the entire hindlimb, which was an average of 3 consecutive 1 mm sections. The tibia bone was used as a marker for hindlimb positioning to ensure analysis was preformed on similar sections for each mouse.

### 6-Week Old Crisis Phase Study

3-Week-old male mdx4cv mice were started on P407 and saline treatments of 6.6 gram P407 or saline per gram animal body weight (BW) of a 10% P407/saline solution administered every 4 days post ween for 3 weeks. It has been shown that the mdx endures a crisis phase of rapid, measurable muscle degeneration from p15 to p42.

### Functional Studies

### 20-Week-Old Exercise Study

10-Week-old male mdx4cv mice were started on P407 and saline treatments of 8.0 gram P407 or saline per gram animal (BW) of a 10% P407/saline solution administered every 3 days for 10 weeks.

### Functional Studies

### Downhill Treadmill Running

The mice were run on a treadmill (Columbus Instruments DETAILS) at a constant 16 m/min rate with a downward inclination of 45° once a week for an average distance of 64 meters per week for 9 weeks to induce muscle damage. During the 10^{th} week, the mice were run for three consecutive days prior to sacrifice immediately following the last running bout on the third day. (Brusse 1997)

### Activity Analysis

In an effort to account for the innate activity levels of the mice, total overnight activities were recorded using Opto M3 animal activity meters (Columbus Instruments) for each mouse, and the mice were grouped into treatment cohorts based on these results. Over the course of the first 9 weeks of running, total overnight activity was assessed post downhill run.

Immediately following the running session on the second day of the 10^{th} week, the mice were again placed into activity monitors along with no run/no treatment age-matched controls and total activity was recorded at 5-minute intervals for the first one hour of recovery.

### 6-Month-Old Long-Term Study

3-Week-old male mdx4cv mice were started on P407 and saline treatments of 8.0 gram P407 or saline per gram animal (BW) of a 10% P407/saline solution administered every 3 days for 5 months.

### Functional Assays

Grip Strength: 5 trials; Adjusted on BW.

A force transducer (Columbia Instruments) allows forelimb grip strength to be tested in a tug of war fashion with the point of grip release is considered the maximum strength. The mouse is carefully handled and pulled by his lower body while his forelimbs are holding onto a custom mouse-designed grid grip handle apparatus. Five trials are performed in a given bout of grip strength testing.

### Post Grip Strength Recovery:

Spontaneous cage activity is monitored with activity meter in two minute intervals for 30 minutes following Grip strength testing.

### Exercise Performance on Voluntary Mouse Wheels:

4″ petsmart mouse wheels hooked up with Sigma Bicycle computers can track speeds and distances run by mice. The mice appear to enjoy the enrichment/exercise and eagerly run at night for up to 4-10 kilometers. The mdx mice run voluntarily 3-8 km every night over their entire lifespan.

### Forced 5% Downhill Treadmill Running:

Run 7.5 minutes for 7.5 m/minute pace then 7.5 additional minutes at 10 m/min pace. It was reported that this is an ideal protocol to cause eccentric muscle damage to challenge the muscle function and performance of the mdx mouse. With treadmill run to exhaustion at speeds of up to 10 m/min, one can readily assess the exercise performance of the mice as well as evaluate their time to recovery after exhaustion.

Forced Downhill Running: 45° slope; 16 m/min;

Mice run in a group and all stop when 1^{st} mouse showed fatigue.

### Post Run Downhill Running Recovery:

Spontaneous cage activity is monitored with activity meters in two minute intervals for two hours.

### Gait Analysis

A series of small chambers is located on top of the treadmill unit, and mice are placed into the chamber that is located directly above the camera so that their gait can be monitored. Recording starts at a slow speed, i.e. the treadmill is slowly turned to 23.4 cm/sec and the walking motion of the mouse is video recorded. The taped recording is subsequently analyzed to detect potential abnormalities in different aspects of their gait. Standard gait indices that are analyzed, include stride, stance and swing time (stride time: time between consecutive paw placements; swing time: the time a paw spends fully in the air; stance time: the time a paw is in contact with the treadmill surface). The gait analysis device has been fully validated and the results have been published (Wooley et al., 2005, Muscle Nerve, 32, (1), 43-50). An alternative method is paw-blots where a non-toxic children's Crayola color paint is blotted on mice paws and their foot prints are made while they run on butcher paper through a dark tunnel.

### Activity Meters or Motion Beam Detectors:

To quantify locomotor activity, experimental mice and control littermates were placed in individual automated photocell activity cages (29×50 cm) with twelve 2 cm high infrared beam detectors in a 4×8 grid (Columbus Instruments, Columbus, Ohio). Mice are habituated and recordings are then made only during their active dark 12 hour cycle per CCM routine. Data is expressed as % of average baseline of control littermates.

Nocturnal Video Surveillance with Infrared Camera:

A Sony camcorder is positioned on tripod to monitor up to 8 cages in stacked 4×2 configuration using the “night vision” infrared feature. The infrared light seems to be invisible to them, but it shows up on a color TV as a monochrome greenish light.

### Biochemical Assays

Creatine Kinase & Cholesterol Levels

Hydroxyproline Level Assay (collagen)

### Poloxamer 407 Pharmacokinetics Information:

Rats received 0.33 g/kg/day (10% w/w solution) P407 i.p.

No significant changes in spleen, liver, or body weight
Increase in white blood cells
Rats received 300 mg P407 i.p.
   Clearance: 0.014 mL/min
   Amount in Urine: 76.3 mg in 24 hrs
   Amount in Liver: 15.9 mg
   Amount in Kidney: 3.1 mg

### (Peer-Reviewed Work by Johnston Tp and Li C)

Rats received 7.5 mg P407 i.p.
   Clearance: 0.00035 mL/min
   Amount in Urine: 1.9075 mg in 24 hrs
   Amount in Liver: 0.3975 mg
   Amount in Kidney: 0.0775 mg

### Absorption

Intraperitoneally

### Distribution

| | | P407 | |
|---|---|---|---|
| Organ | DI H20 | Autofluorescence | P407 tag |
| Brain | − | − | − |
| Heart | − | − | + |
| Kidney | − | − | + |
| Liver | − | − | − |
| Spleen | − | − | + |

### Metabolism

The literature suggests that block copolymers are not metabolized and are excreted unchanged in the urine and feces.

### Excretion

Rats received 300 mg P407 i.p.
   Clearance: 0.014 mL/min
   Amount in Urine: 76.3 mg in 24 hrs
   Amount in Liver: 15.9 mg
   Amount in Kidney: 3.1 mg
Rats received 7.5 mg P407 i.p. (standard weight: 25 g)
   Clearance: 0.00035 mL/min
   Amount in Urine: 1.9075 mg in 24 hrs
   Amount in Liver: 0.3975 mg
   Amount in Kidney: 0.0775 mg

A 0.0825 ml container of P407 is put into a 2 ml of blood. The Vd of the P407 is 2.0825 ml. If, each minute, you empty 0.00035 ml of the P407 into the 0.0825 ml container, discard this, and replace it with 0.0825 ml of water. The clearance is 0.0825 ml per minute. The kel is Cl/Vd=0.0825/2.0825=0.0396158. The elimination half life is: t1/2=0.693×Vd/Cl=(0.693)(2.0825/0.0825)=17.493. The elimination half life is 17.493 min.

Throughout this application, author and year, and patents, by number, reference various publications, including United States patents. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

### REFERENCES

Ahmed, F. Alexandridis, P., Neelamegham, S., “Synthesis and Application of Fluorescein Labeled Pluronic Block Copolymers to Study Polymer-Surface Interactions”, Langmuir, 17: 537-546, 2001.
Fussnegger B., Poloxamers (2) Lutrol® F 127 (Poloxamer 407). BASF ExAct April; 4:7-9 (2000).
Ghebeh H, Handa-Corrician A, Butler M. Development of an assay for the measurement of the surfactant pluronic F-68 in mammalian cell culture medium. Anal Biochem. 1998 Aug. 15; 262(1):39-44.
Hoffman E P, Brown R H Jr, Kunkel L M. Dystrophin: the protein product of the Duchenne muscular dystrophy locus. Cell. December 24; 51 (6):919-28 (1987).
Im W B, Phelps S F, Copen E H, Adams E G, Slichtom J L, Chamberlain J S. Differential expression of dystrophin isoforms in strains of mdx mice with different mutations. Hum Mol Genet. August; 5(8):1149-53 (1996).
Johnston T P, Miller S C. Toxicological evaluation of poloxamer vehicles for intramuscular use. J Parenter Sci Technol. March-April; 39(2):83-9 (1985).
Johnston T P. The P-407-induced murine model of dose-controlled hyperlipidemia and atherosclerosis: a review of findings to date. J Cardiovasc Pharmacol. April; 43(4):595-606 (2004).
Khattak S F, Bhatia S R, Roberts S C. Pluronic F127 as a cell encapsulation material: utilization of membrane-stabilizing agents. Tissue Eng. May-June; 11 (5-6):974-83 (2005).
Lee R C, River L P, Pan F S, Ji L, Wollmann R L. Surfactant-induced sealing of electropermeabilized skeletal muscle membranes in vivo. Proc Natl Acad Sci USA. 1992 May 15; 89(10):4524-8.
Lutrol® F 127. BASF Technical Information. August (2002).
Maskarinec S A, Hannig J. Lee R C, Lee K Y. Direct observation of poloxamer 188 insertion into lipid monolayers. Biophys J. 2002 March; 82(3):1453-9.
Palmer W K, Emeson E E, Johnston T P. Poloxamer 407-induced atherogenesis in the C57BL/6 mouse. Atherosclerosis. January; 136(1):115-23 (1998).
Vilguin J T, Brussee V, Asselin I, Kinoshita I, Ginciras M, Tremblay J P. Evidence of mdx mouse skeletal muscle fragility in vivo by eccentric running exercise. Muscle Nerve. 1998 May; 21(5):567-76.
Wasan K M, Subramanian R, Kwonci M, Goldberci I J, Wriciht T, Johnston T P. Poloxamer 407-mediated alterations in the activities of enzymes regulating lipid metabolism in rats. J Pharm Pharm Sci. May-August; 6(2):189-97 (2003).
Wout Z G, Pec E A, Maciciiore J A, Williams R H, Palicharla P, Johnston T P. Poloxamer 407-mediated changes in plasma cholesterol and triglycerides following intraperitoneal injection to rats. J Parenter Sci Technol. November-December; 46(6):192-200 (1992).
Wu G, Majewski J, Ege C, Kjaer K, Weygland M J, Lee K Y. Lipid corralling and poloxamer squeeze-out in membranes. Phys Rev Lett. July 9; 93(2):028101 (Epub Jul. 7, 2004).
Yasuda S, Townsend D, Michele D E, Favre E G, Day S M, Metzger J M. Dystrophic heart failure blocked by membrane sealant poloxamer. Nature. August 18; 436(7053):1025-9 (Epub Jul. 17, 2005).
J. Quinlan, B. Wong, R. Niemeier, A. McCulloucih, L. Levin, M. Emanuele. Poloxamer 188 failed to prevent exercise-induced membrane breakdown in mdx skeletal muscle fibers. Neuromuscular Disorders, Volume 16, Issue 12, Pages 855-864.
Frey S L, Zhanci D, Caricinano M A, Szleifer I, Lee K Y. Effects of block copolymer's architecture on its association with lipid membranes: Experiments and simulations. J Chem. Phys. 2007 Sep. 21; 127(11):114904. PMID: 17887877 [PubMed—in process]
Frey S L, Lee K Y. Temperature dependence of poloxamer insertion into and squeeze-out from lipid monolayers. Langmuir. 2007 Feb. 27; 23(5):2631-7. PMID: 17309214 [PubMed—indexed for MEDLINE]
Maskarinec S A, Hannig J, Lee R C, Lee K Y. Direct observation of poloxamer 188 insertion into lipid monolayers. Biophys J. 2002 March; 82(3):1453-9. PMID: 11867460 [PubMed—indexed for MEDLINE]
Grindel J M, Jaworski T, Piraner O Emanuele R M, Balasubramanian M. Distribution, Metabolism and Excretion of a Novel Surface-Active Agent, Purified Poloxamer 188, in Rat, Dog and Man. [Personal Communication].
Rodriguez S C, Sincier E J. Toxicology of polyoxyalkylene block copolymers. In Non-Ionic Surfactants: Polyoxyalkylene Block Copolymers, Nace V M (ed). Marcel Dekker New York, 1996; 211-241.

P407 IP Injection Toxicity?

| | | | | | |
|---|---|---|---|---|---|
| 3 Weeks | 3 Weeks | 10 Weeks | 10 Weeks | 5 Month | 5 Months |
| Control | on P407 | Control | on P407 | Control | on P407 |

| | | | | |
|---|---|---|---|---|
| Body | 18.5 g ± 2.02 | 19.4 g ± 2.51 | 30.5 g ± 3.21 | 31.3 g ± 3.11 |
| Weight | | | | |
| Morbidity | 2 | 0 | 1 | 1 |

Other info here for Bodyweights, morbidity and mortality, must talk about cholesterol, belly P407 accumulation, dermatitis/alopecia.

What is claimed is:

1. A molecular sealant comprising a therapeutically effective amount of a poloxamer capable of sealing a cell or tissue from leaks.

2. The sealant according to claim 1, wherein said poloxamer is poloxamer 407.

3. The sealant according to claim 1 for use in treating skeletal muscle cells.

4. A method of treating tissue leakages by applying the sealant of claim 1 to the tissue in need of treatment.

5. A method of treating cellular leakages by applying the sealant of claim 1 to the cells in need of treatment.

6. A composition for treating cellular leakages comprising a poloxamer capable of sealing a cell from leaks in a pharmaceutically acceptable carrier.

7. The composition according to claim 6, wherein said poloxamer is poloxamer 407.

8. The composition according to claim 6, wherein said composition is formulated for multiple methods of administration.

9. The composition according to claim 8, wherein said methods of administration are selected from the group consisting essentially of intraperitoneal, intramuscular, orally, intravenous, and topically.

10. A composition for treating cellular leakages comprising a poloxamer capable of sealing a tissue from leaks in a pharmaceutically acceptable carrier.

11. The composition according to claim 10, wherein said poloxamer is poloxamer 407.

12. The composition according to claim 10, wherein said composition is formulated for multiple methods of administration.

13. The composition according to claim 12, wherein said methods of administration are selected from the group consisting essentially of intraperitoneal, intramuscular, orally, intravenous, and topically.
